(19) United States
(12) Patent Application Publication
(10) Pub. No.: US 2022/0265405 A1
(43) Pub. Date: Aug. 25, 2022

(54) METHOD AND APPARATUS FOR APPLICATION OF A SUBSTANCE

(71) Applicant: Christopher Robert Martin, Giralang (AU)

(72) Inventor: Christopher Robert Martin, Giralang (AU)

(21) Appl. No.: 17/631,802

(22) PCT Filed: Jul. 29, 2019
(86) PCT No.: PCT/AU2019/050791

§ 371 Date: Jan. 31, 2022

(30) Foreign Application Priority Data
Jul. 30, 2018 (AU) 2018902743

(51) Int. Cl. A61C 17/02; A61C 17/022; A61F 13/40; A61C 17/22; A46B 13/04

(52) U.S. Cl. CPC A61C 17/0202; A61C 17/022; A61M 35/006; A61C 17/221; A46B 13/04; A61M 2205/587; A61M 2205/8206

(57) ABSTRACT

Embodiments of the present disclosure provides oral applicators and methods for applying a substance to a site inside a mouth cavity of a human or an animal. An oral applicator includes a body adapted for being held by a human hand. The oral applicator further includes an applicator head extending from the body. The applicator head also includes an applicator surface distally located in relation to the body and adapted for use in applying the substance to the site. Further, the oral applicator includes a gas stream generator present near the applicator head for directing a stream of gas from the applicator head. The site may be dried during at least one of prior to, during and after an application of the substance. The stream of gas may be directed via an outlet proximate to the applicator surface.

### TECHNICAL FIELD

The present disclosure is related to the field of medical devices. More particularly, the present disclosure relates to an apparatus for and a method of applying a substance to a lesion site in the mouth cavity of a person or animal.

### BACKGROUND

Any references to methods, apparatus or documents of the prior art are not to be taken as constituting any evidence or admission that they formed, or form part of the common general knowledge.

Many suffer from the discomfort caused by mouth lesions. Often, these are treatable with topically applied medicaments in the form of creams, pastes, gels and liquids. Common examples of such lesions are mouth ulcers.

To apply the medicament requires some alacrity. Often it can be a hit- and miss process because the person attempting application is unable to see the exact location of the site requiring treatment. Using the fingers carries with it the risk of causing infection by introducing pathogens. The hand may block vision of the site and prevent illumination by a flashlight.

Furthermore, the site of application, being in the mouth is usually wet and not susceptible to the successful adhesion of a treatment substance.

### SUMMARY

It is an object of this invention to address the shortcomings of the prior art and, in doing so, to provide a substance applicator that has means for drying the site of application or the substance applied and is suitable for use in a user's mouth cavity.

The preceding discussion of the background to the invention is intended to facilitate an understanding of the present invention. However, it should be appreciated that the discussion is not an acknowledgement or admission that any of the material referred to was part of the common general knowledge in Australia or elsewhere as at the priority date of the present application.

Further, and unless the context clearly requires otherwise, throughout the description and the claims, the words ‘comprise’, ‘comprising’, and the like are to be construed in an inclusive sense, meaning “including, but not being limited to as opposed to an exclusive or exhaustive sense, meaning “including this and nothing else”.

An embodiment of the present disclosure provides an oral applicator adapted for applying a substance to a site inside a mouth cavity of a human or an animal. The oral applicator includes a body adapted for being held by a human hand. The oral applicator further includes an applicator head extending from the body. The applicator head also includes an applicator surface distally located in relation to the body and adapted for use in applying the substance to the site. Further, the oral applicator includes a gas stream generator present near the applicator head for directing a stream of gas from the applicator head. The site may be dried during at least one of prior to, during and after an application of the substance. The stream of gas may be directed via an outlet proximate to the applicator surface and the substance is applied via at least one of the application surface and a pad attached to the application surface.

According to an aspect of the present disclosure, the body defines a housing for housing the gas stream generator.

According to another aspect of the present disclosure, the applicator head is of elongate dimensions and may be in a shape of an elbow.

In some embodiments, the gas stream generator includes a fluid conduit from a source of gas under pressure to the outlet proximate to the applicator surface.

According to another aspect of the present disclosure, the gas stream generator includes a blower.

In some embodiments, the blower comprises a fan and means for driving the fan.

The fan driving means may include a motor and a power source operatively connected to the motor. The blower may further include a power switch operable to switch power to the fan motor on and off.

According to another aspect of the present disclosure, the blower may include an air intake to the fan, the intake passing through the body.

According to another aspect of the present disclosure, the applicator head is detachably connected to the body by connector (or connector means) including a socket.

According to another aspect of the present disclosure, the socket connects a fluid conduit from the fan of the gas stream generator (or in the housing) with a fluid conduit in the applicator head leading to the outlet.

According to another aspect of the present disclosure, the applicator surface includes the pad including a resilient pad. Preferably, the resilient pad is configured to absorb.

In some embodiments, the pad may be a sponge-like material.

According to another aspect of the present disclosure, the resilient pad has a contiguous surface. In some embodiments, the contiguous surface is preferably contoured. In some embodiments, the contiguous surface is rounded.

According to another aspect of the present disclosure, the pad is planar.

In some embodiments of the present disclosure, the pad may have a shape other than generally planar, for example rounded, including substantially hemispherical.

According to another aspect of the present disclosure, the resilient pad is configured to be detached from the applicator surface. Preferably, the resilient pad is replaceable with another pad of a differing surface configurations.

According to another aspect of the present disclosure, the oral applicator includes an illuminator operable for illuminating the site to which a substance is to be applied using the applicator surface. The illuminator (also referred as illumination means) may include a light pipe leading light from a light source to a light outlet on the applicator head.

According to another aspect of the present disclosure, the gas stream generator includes a reservoir of gas under pressure and means for allowing gas to escape from the reservoir in a stream. In some embodiments, the reservoir stores the gas in an aerosol form.

According to another aspect of the present disclosure, the applicator head is detachably connected to the body.

Another embodiment of the present disclosure provides a method for applying a substance to a site in a mouth cavity of a human or an animal. The method includes providing an oral applicator comprising an applicator head extending from a body adapted for being held by a human hand, wherein the applicator head comprises an applicator surface distally located in relation to the body and adapted for use in applying the substance to the site, and a gas stream generator present near the applicator head for directing a stream of gas from the applicator head on to the site. The method also includes applying said substance to application surface of the applicator head and directing a stream of the gas for drying the site. The stream of gas may be directed via an outlet proximate to the applicator surface. The method further includes applying the substance from the applicator surface to the site for transferring the substance to the site.

According to an aspect of the present disclosure, the method also includes directing the stream of the gas for drying the site during at least one of prior to application of the substance, during application of the substance to the site, and after application of the substance to the site.

According to an aspect of the present disclosure, the body defines a housing for housing the gas stream generator. The gas stream generator may include a fluid conduit from a source of gas under pressure to the outlet proximate to the applicator surface. the gas stream generator comprises a blower.

In some embodiments, the method includes providing the gas stream generator in the form of a blower. Preferably, the blower is housed within the body and connected to be in fluid communication with the outlet. The blower may include a fan and means for driving the fan.

According to an aspect of the present disclosure, the applicator head comprises an applicator surface on to which the substance is applied for transfer to the site.

According to an aspect of the present disclosure, the substance is applied via at least one of the application surface and a pad attached to the application surface.

According to a yet another aspect of the present disclosure, there is provided a kit for applying a substance to a site inside the mouth cavity of a human or an animal, the kit comprising applicator apparatus comprising: a body adapted for being held by a person's hand, an applicator head extending from the body, the applicator head comprising an applicator surface distally located in relation to the body and adapted for use in applying a substance to the site, and gas stream generator configured for directing a stream of gas from the applicator head on to the site, whereby the site may be dried before or after application of the substance.

A yet another embodiment of the present disclosure provides a kit for applying a substance to a site inside a mouth cavity of a human or an animal. The kit includes a body adapted for being held by a human hand. The kit also includes an applicator head detachably connected to the body. The applicator head may include an applicator surface distally located in relation to the body and adapted for use in applying the substance to the site. The kit also includes a gas stream generator present near the applicator head for directing a stream of gas on to the site for drying the site. The site may be dried before, during, or after application of the substance. Further, the gas stream generator may include a fluid conduit from a source of gas under pressure to an outlet proximate to the applicator surface.

According to an aspect of the present disclosure, the kit includes a plurality of applicator heads configured to interchangeably attach to the body.

According to another aspect of the present disclosure, the kit includes a plurality of applicator elements including interchangeable respective applicator surfaces for operatively fitting to the applicator head.

According to another aspect of the present disclosure, the applicator surfaces are of different respective configurations.

According to another aspect of the present disclosure, the gas stream generator includes a blower. The blower may include a fan and means for driving the fan.

According to another aspect of the present disclosure, the applicator surface may include a resilient pad configured to absorb. The resilient pad may include a contiguous surface.

According to another aspect of the present disclosure, the resilient pad is configured to be detached from the applicator surface.

According to another aspect of the present disclosure, an illuminator operable for illuminating the site to which a substance is to be applied using the applicator surface. In some embodiments, the illuminator may include a light pipe leading light from a light source to a light outlet on the applicator head.

In some embodiments, the substance is applied via at least one of the application surface and the resilient pad attached to the application surface.

In some embodiments, the kit includes a rechargeable battery cell.

### DETAILED DESCRIPTION

Preferred features, embodiments and variations of the invention may be discerned from the following detailed description which provides sufficient information for those skilled in the art to perform the invention. The detailed description is not to be regarded as limiting the scope of the preceding summary of the invention in any way.

Specific embodiments of the present invention are described, by way of example only, with reference to the accompanying drawings, in which:

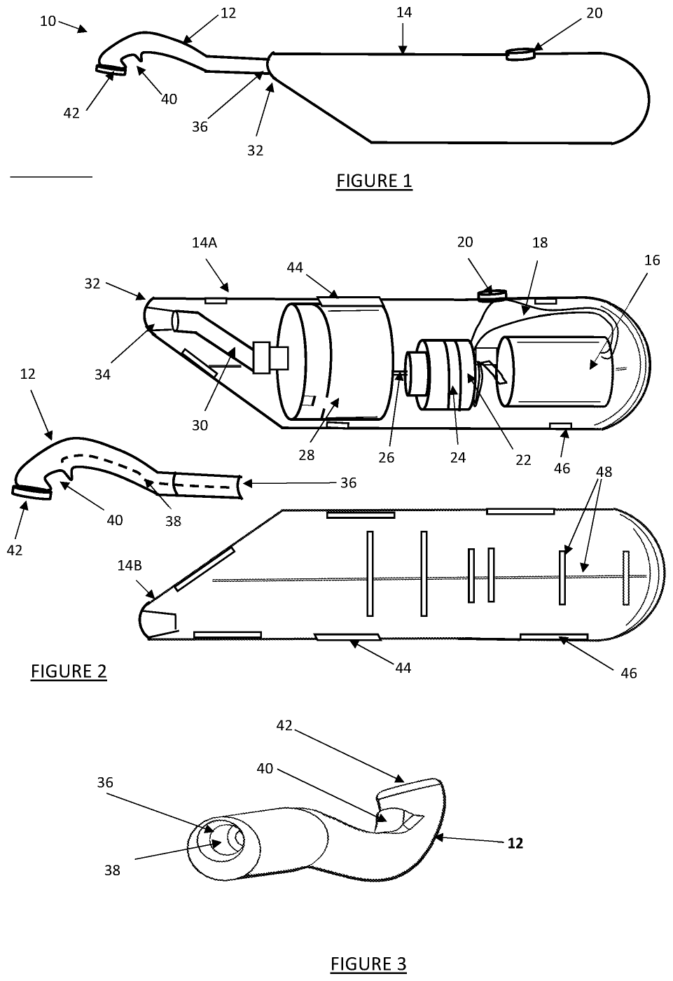

FIG. 1 shows in schematic side view of an exemplary oral applicator 10 (hereinafter may also be referred as an applicator apparatus without change in its meaning), in accordance with an embodiment of the present disclosure;

FIG. 2 is a side view of the oral applicator 10 of the FIG. 1 with housing opened to show internal components, in accordance with an embodiment of the present disclosure; and

FIG. 3 illustrates a perspective view of an applicator head assembly of the oral applicator 10 of the FIG. 1 detached from the housing.

Prior to discussing the details of the present disclosure, it should be understood that several sections of the following description are presented largely in terms of logic and operations that may be performed by conventional components. These components, which are grouped in a single location being the body and applicator extension of the invention, generally include a fan, a motor driving the fan, a power source for the motor and fluid conduits. To the extent the components may be or are distributed, the components are accessible to each other via communication links. In the following description, numerous specific details are set forth in order to provide a description of the present disclosure. It will be apparent to one skilled in the art, however, that the disclosed subject matter may be practiced without some or all of the specific details.

Referring now to the drawings, wherein like reference numbers are used herein to designate like elements throughout, the various views and embodiments of the applicator apparatus of the invention are illustrated and described, and other possible embodiments are described. The figures are not necessarily drawn to scale, and in some instances the drawings have been exaggerated and/or simplified in places for illustrative purposes only. One of ordinary skill in the art will appreciate the many possible applications and variations based on the following examples of possible embodiments.

The embodiment below is described in the context of use on a human with a mouth lesion. However, it should not be construed as limited to a lesion or to human use. It may also be used on animals of various kinds and reduces the risk of biting injuries.

Referring to the FIG. 1, a schematic side view of the oral applicator 10, in accordance with an embodiment of the present disclosure is shown. The oral applicator 10 may be adapted for applying a substance to a site inside a mouth cavity of a user such as, but not limited to, a human or an animal. Hereinafter the oral applicator 10 may also be referred as a kit 10, an applicator 10 or an applicator apparatus 10 without change in its meaning. The oral applicator 10 has an applicator head assembly 12 (hereinafter, may also be referred as an applicator head), which is detachably attached to a body 14 (hereinafter may also be referred as an applicator body 14) by means of connector like a socket. The body 14 is adapted for being held by a user hand like a human hand. The body 14 is generally tubular and defines an internal space which houses inner working components of the oral applicator 10.

The applicator head assembly 12 may extends from the body 14. In some embodiments, the applicator head assembly 12 may include an applicator surface 42 distally located in relation to the body and adapted for use in applying the substance to the site. The site may be a treatment site or application site where a substance needs to be applied. As shown, the head assembly 12 is of elongate dimensions and is in a shape of an elbow, rendering it suitable for insertion into a recipient's (i.e. user's) mouth cavity, while the body 14 is held substantially completely outside. The shape of the head assembly 12 may ensure minimal obscuring of the interior of the mouth for the person i.e. the recipient using it.

The oral applicator 10 may also include a gas stream generator present near the applicator head assembly 12 for directing a stream of gas from the applicator head assembly 12 on to the site. The site may be dried prior to an application of the substance and/or during the application of the substance and/or after the application of the substance. The body 14 may define a housing for housing the gas stream generator.

In some embodiments, the applicator surface 42 may include a resilient pad. The resilient pad may be configured to absorb. Further, the resilient pad may include a contiguous surface. In some embodiments, the contiguous surface is rounded. Further, the resilient pad is configured to be detached from the applicator surface 42. In some embodiments, the resilient pad is replaceable with another pad of a differing surface configuration.

Referring to FIG. 2, a side view of the oral applicator 10 of the FIG. 1 with housing opened to show internal components of the oral applicator 10 is shown. As shown, the applicator body 14 includes two separable half-pieces 14A, and 14B, which may be clipped together for use, utilizing known connection methods and formations 46. When the half-pieces 14A, and 14B are operatively connected to form the housing, it is suitably shaped to be comfortably held in the hand of a user. In some embodiments, the half-pieces 14A and 14B of the applicator body 14 are made of a moulded plastics material, for example polypropylene. Any other suitable material may be used to make the half-pieces 14A and 14B.

As discussed with reference to the FIG. 1, the gas stream generator may include a fluid conduit from a source of gas under pressure to an outlet 40 (may also be referred as an air or gas outlet) proximate to the applicator surface 42. Further, the gas stream generator may include a blower housed within the body. The blower is connected to be in fluid communication with the outlet 40. The blower may include a fan and means for driving the fan. The fan driving means includes a motor 22 and a power source operatively connected to the motor 22. The blower may include a power switch 20 operable to switch power to the motor 22 (may also be referred as the fan motor 22 without change in its meaning) on and off.

The applicator head 12 (i.e. the applicator head assembly 12) is detachably connected to the body 14 by a connector including a socket 34. The socket 34 may connect a fluid conduit from the fan of the gas stream generator to a fluid conduit in the applicator head 12 leading to the outlet 40.

Further, the oral applicator 10 includes a rechargeable and replaceable battery cell 16. The battery cell 16 is supported by a mouldings 48 with the housing at a lower end of body 14. The battery cell 16 can be replaced with fresh cells when flat. The battery cell 16 may be rechargeable battery cell. In some embodiments the battery cell 16 are solar battery cell 16. The battery cell 16 may be operatively connected by a wiring 18 to the switch 20 (may be referred as a fan switch 20) which, when pushed, connects or disconnects electric current to the motor 22.

The motor 22 is operatively connected by a drive shaft 26 to drive the fan, which is operatively mounted within a fan housing 28. The motor 22 is secured to the half piece 14A of the applicator body 14 by a retaining bracket 24.

The fan takes in air via an intake 44 passing though the half-piece 14A and expels the air under pressure in an accelerated stream through an outlet duct 30, which directs the airflow to an outlet at an end 32 of the applicator body 14. The outlet defines the socket 34 into which a ducted end 36 of applicator head assembly 12 is inserted, to provide fluid communication between the body 14 and the applicator head assembly 12.

In some embodiments, the gas stream is directed via the outlet 40 and the substance is applied via the application surface 42 or the pad attached to the application surface.

Referring now to the FIG. 3, a perspective view of the applicator head assembly 12 of the oral applicator 10 detached from the housing is shown. In some embodiments, the applicator head assembly 12 is in the form of a ducted elbow-like attachment that funnels air through an internal duct 38 to the outlet 40, located immediately to the rear of the applicator surface 42 or a delivery pad present on the applicator surface 42 (hereinafter, may also be referred as a surface face 42). In some embodiments, the pad may be formed using a porous sponge like material adapted to absorb.

In some embodiments, a substance may be applied to the applicator surface 42 for transferring the substance to the treatment site. Alternatively, a delivery pad including a resilient pad is attached to the applicator surface 42. In such embodiments, the substance may be applied to the delivery pad for applying to the treatment site in a recipient's mouth cavity. The delivery pad may be a resilient pad configured to absorb. The delivery pad may include a contiguous surface. In some embodiments, the contiguous surface is rounded. The delivery pad is configured to be detached from the applicator surface. In some embodiments, the delivery pad is replaceable with another pad of a differing surface configuration.

In some embodiments, the gas stream generator includes a reservoir of gas under pressure and means for allowing the gas to escape from the reservoir in a stream. The gas may be air or any other suitable gas. In some embodiments, the reservoir stores the gas in an aerosol form.

In some embodiments, the outlet 40 (hereinafter, may also be referred as air leaving outlet 40) may be shaped and oriented to direct an emerging stream of air onto the surface in a recipient's mouth at the site where the substance is to be or is being applied. In some embodiments, the outlet 40 is configured to direct the stream of the gas for drying the site prior to application of the substance and/or during application of the substance to the site, and/or after application of the substance to the site.

The substance may be of any composition suitable for application to a site in the mouth cavity. In an example, the substance is a medicament for treating a mouth condition. The condition may be sited on the tongue, a gum or a tooth, the palette, or a portion of the mouth lining, and so forth. Creams or gels in a semi solid form are applied to the applicator surface 42 of the head assembly 12. The applicator surface 42 may transmit creams and medications in semi solid form directly onto the receiving surface.

To dry the site prior to application during the application, and/or to dry the substance being applied, after its application or, the user, such as a doctor or the recipient, may activate the fan while holding the body and move the apparatus so that the air leaving outlet 40 is directed to the desired area and for required duration.

In a further embodiment, the applicator surface 42 may be in the form of an absorbent pad, for example a soft sponge that can be impregnated with a liquid medication. The sponge may transmit the medication to the receiving site when gently compressed against it by a dabbing action by the user of the apparatus.

In an alternative embodiment, the apparatus further comprises a light source i.e. an illuminator operable for illuminating the site to which a substance is to be applied using the applicator surface 42. The illuminator may include a light pipe leading light from a light source to a light outlet on the applicator head assembly 12. In some embodiments, the source may be a light-emitting diode (LED) located at the operative distal end of the applicator head assembly 12 and connected electrically to the power source using circuitry of known type and a connector associated with the connecting the socket 34 of the body 14 with a socket 36 of the head assembly 12. The LED may be connected for activation by means of the fan switch 20, so that fan and LED are simultaneously powered. In some embodiments, the LED may have a dedicated separate switch, allowing the user the choice of using the light and fan independently or simultaneously. The light source assists the user in observing the interior of the mouth and the site for application of the substance.

In an embodiment, the LED is housed within the body of the apparatus and is connected by a light pipe to a light outlet on the applicator head. The light outlet may be positioned on the leading end of the applicator, for example just above applicator pad. The light would be transmitted via the light pipe and through the elbow of applicator head assembly 12, to illuminate the receiving surface.

In some embodiments, the elbow shaped head assembly 12 is made of a see-through material, for example a Perspex formulation. When the elbow shaped head assembly 12 is attached to be in communication with a light source transmitting light from within the applicator body 14, the light is emitted into the mouth cavity via the elbow attachment. It is also within the scope of the invention for a portion, rather than the whole of the applicator assembly 12 to be a light conduit to the treatment site.

In an embodiment, the battery cell 16 may be re-chargeable, eliminating the need to be removed. A recharge connection point may be included at an appropriate site on the surface of the applicator body 14, for example at a bottom end of the applicator body, for connection with an external recharger.

It will be appreciated that instead of polypropylene, the applicator body 14 may be manufactured or formed from a different plastics material, or any other suitable substance, include from a metal alloy or ceramic composite. The body shape may be altered in other embodiments to provide any suitably eye-catching design, provided it remains practically shaped for ease of holding by the user.

It is considered that for practical reasons, air is the gas of choice for use in drying the application site. However, it is still within the scope of this invention to provide means for expelling an alternative gas or mixtures of gases selected for the drying of the application/treatment site prior to application of the substance, or to dry the substance once applied. Thus, certain gases may be selected when a particular substance demands it, for example if the substance is required to undergo a reaction after application to the site. In some embodiments, the gas may be made available in aerosol form. The gas of choice may be supplied from a mini-canister mounted in the housing and connected to the supply conduit. A hand-activated valve is connected in line to control the supply outlet from the canister.

In alternative embodiments, the body 14 and applicator head assembly 12 are not separable, but manufactured in a single piece. This means the body 14 and applicator head assembly 12 may form a unitary structure. The interior is accessible through a removable closure.

According to an embodiment of the present disclosure provides an oral applicator adapted for applying a substance to a site inside a mouth cavity of a human or an animal. The oral applicator includes a body adapted for being held by a human hand. The oral applicator further includes an applicator head extending from the body. The applicator head also includes an applicator surface distally located in relation to the body and adapted for use in applying the substance to the site. Further, the oral applicator includes a gas stream generator present near the applicator head for directing a stream of gas from the applicator head. The site may be dried during at least one of prior to, during and after an application of the substance. The stream of gas may be directed via an outlet proximate to the applicator surface and the substance is applied via at least one of the application surface and a pad attached to the application surface.

According to an aspect of the present disclosure, the body defines a housing for housing the gas stream generator.

According to another aspect of the present disclosure, the applicator head is of elongate dimensions and may be in a shape of an elbow.

In some embodiments, the gas stream generator includes a fluid conduit from a source of gas under pressure to the outlet proximate to the applicator surface.

According to another aspect of the present disclosure, the gas stream generator includes a blower.

In some embodiments, the blower includes a fan and means for driving the fan.

The fan driving means may include a motor and a power source operatively connected to the motor. The blower may further include a power switch operable to switch power to the fan motor on and off.

According to another aspect of the present disclosure, the blower may include an air intake to the fan, the intake passing through the body.

According to another aspect of the present disclosure, the applicator head is detachably connected to the body by connector (or connector means) including a socket.

According to another aspect of the present disclosure, the socket connects a fluid conduit from the fan of the gas stream generator (or in the housing) with a fluid conduit in the applicator head leading to the outlet.

According to another aspect of the present disclosure, the applicator surface includes a resilient pad. Preferably, the pad is configured to absorb.

In some embodiments, the pad may be a sponge-like material.

According to another aspect of the present disclosure, the resilient pad has a contiguous surface. In some embodiments, the contiguous surface is preferably contoured. In some embodiments, the contiguous surface is rounded.

According to another aspect of the present disclosure, the pad is planar.

In some embodiments of the present disclosure, the pad may have a shape other than generally planar, for example rounded, including substantially hemispherical.

According to another aspect of the present disclosure, the resilient pad is configured to be detached from the applicator surface. Preferably, the resilient pad is replaceable with another pad of a differing surface configurations.

According to another aspect of the present disclosure, the oral applicator includes an illuminator operable for illuminating the site to which a substance is to be applied using the applicator surface. The illuminator (also referred as illumination means) may include a light pipe leading light from a light source to a light outlet on the applicator head.

According to another aspect of the present disclosure, the gas stream generator includes a reservoir of gas under pressure and means for allowing gas to escape from the reservoir in a stream.

According to another aspect of the present disclosure, the applicator head is detachably connected to the body.

According to another aspect of the present disclosure, there is provided a method of applying a substance to a receiving site in the mouth of a person or animal, the method comprising steps of: providing apparatus comprising an applicator head connected to a body adapted for holding by a human hand and gas stream generator configured for directing a stream of gas from the extension on to the site, applying said substance to a portion of the applicator head, directing a stream of the gas to cause drying of the site, and applying the applicator head to the site to transfer at least some of the substance to the site.

According to another embodiment of the present disclosure, there is provided an oral surface applicator adapted for applying a substance to a site inside the mouth cavity of a human or an animal, the applicator comprising a body adapted for being held by a person's hand, an applicator head extending from the body, the applicator head comprising an applicator surface distally located in relation to the body and adapted for use in applying a substance to the site, and a gas stream generator configured for directing a stream of gas from the applicator head on to the site. The site may be dried before or after application of the substance.

Another embodiment of the present disclosure provides a method for applying a substance to a site in a mouth cavity of a human or an animal. The method includes providing an oral applicator comprising an applicator head extending from a body adapted for being held by a human hand. The applicator head may include an applicator surface distally located in relation to the body and adapted for use in applying the substance to the site. The oral applicator also includes a gas stream generator present near the applicator head for directing a stream of gas from the applicator head on to the site. The method also includes applying said substance to application surface of the applicator head and directing a stream of the gas for drying the site. The stream of gas may be directed via an outlet proximate to the applicator surface. The method further includes applying the substance from the applicator surface to the site for transferring the substance to the site.

According to an aspect of the present disclosure, the method also includes directing the stream of the gas for drying the site during at least one of prior to application of the substance, during application of the substance to the site, and after application of the substance to the site.

According to an aspect of the present disclosure, the body defines a housing for housing the gas stream generator. The gas stream generator may include a fluid conduit from a source of gas under pressure to the outlet proximate to the applicator surface. the gas stream generator comprises a blower.

In some embodiments, the method includes providing the gas stream generator in the form of a blower. Preferably, the blower is housed within the body and connected to be in fluid communication with the outlet. The blower may include a fan and means for driving the fan.

According to an aspect of the present disclosure, the applicator head comprises an applicator surface on to which the substance is applied for transfer to the site.

According to a yet another aspect of the present disclosure, there is provided a kit for applying a substance to a site inside the mouth cavity of a human or an animal, the kit comprising applicator apparatus comprising: a body adapted for being held by a person's hand, an applicator head extending from the body, the applicator head comprising an applicator surface distally located in relation to the body and adapted for use in applying a substance to the site, and gas stream generator configured for directing a stream of gas from the applicator head on to the site, whereby the site may be dried before or after application of the substance.

According to an aspect of the present disclosure, the kit includes a plurality of applicator heads configured to interchangeably attach to the body.

According to another aspect of the present disclosure, the kit includes a plurality of applicator elements including interchangeable respective applicator surfaces for operatively fitting to the applicator head.

According to another aspect of the present disclosure, the applicator surfaces are of different respective configurations.

According to another aspect of the present disclosure, the gas stream generator includes a blower. The blower may include a fan and means for driving the fan.

According to another aspect of the present disclosure, the applicator surface may include a resilient pad configured to absorb. The resilient pad may include a contiguous surface.

According to another aspect of the present disclosure, the substance is applied via at least one of the application surface and the resilient pad attached to the application surface.

According to another aspect of the present disclosure, the resilient pad is configured to be detached from the applicator surface.

According to another aspect of the present disclosure, an illuminator operable for illuminating the site to which a substance is to be applied using the applicator surface. In some embodiments, the illuminator may include a light pipe leading light from a light source to a light outlet on the applicator head.

According to another aspect of the present disclosure, the stream of gas is directed via the outlet proximate to the applicator surface and the substance is applied via at least one of the application surface and the resilient pad attached to the application surface.

In some embodiments, the kit includes a rechargeable battery cell.

The disclosed oral applicator includes the device has an LED light source in the device handle so light enters the applicator head to illuminate the inside of the mouth. The removable applicator head assembly includes an air channel through which an air stream generated by an impeller or gas reservoir in the body of the oral applicator passes down the air stream channel to dry the affected area to facilitate the application of the oral cream to the lesion.

These embodiments merely illustrate particular examples of the method, kit and apparatus of the invention providing means for applying a substance to a receiving site in the mouth of a person or animal. It will be appreciated by those skilled in the art and having the benefit of this disclosure, that the drawings and detailed description herein are to be regarded in an illustrative rather than a restrictive manner and are not intended to be limiting to the particular forms and examples disclosed. On the contrary, included—even if not expressly described—are any further modifications, changes, rearrangements, substitutions, alternatives, design choices, and embodiments apparent to or discernible by those of ordinary skill in the art, without departing from the spirit and scope hereof, as defined by the following claims. Thus, it is intended that the following claims be interpreted to embrace all such further modifications, changes, rearrangements, substitutions, alternatives, design choices, and embodiments.

With the insight gained from this disclosure, the person skilled in the art is well placed to discern further embodiments by means of which to put the claimed invention into practice.

In compliance with the statute, the invention has been described in language more or less specific to structural or methodical features. The term “comprises” and its variations, such as “comprising” and “comprised of” is used throughout in an inclusive sense and not to the exclusion of any additional features. It is to be understood that the invention is not limited to specific features shown or described since the means herein described comprises preferred forms of putting the invention into effect. The invention is, therefore, claimed in any of its forms or modifications within the proper scope of the appended claims appropriately interpreted by those skilled in the art.

Throughout the specification and claims (if present), unless the context requires otherwise, the term “substantially” or “about” will be understood to not be limited to the value for the range qualified by the terms.

Any embodiment of the invention is meant to be illustrative only and is not meant to be limiting to the invention. Therefore, it should be appreciated that various other changes and modifications can be made to any embodiment described without departing from the spirit and scope of the invention.

What is claimed is:

1. An oral applicator adapted for applying a substance to a site inside a mouth cavity of a human or an animal, the oral applicator comprising
a body adapted for being held by a human hand;
an applicator head extending from the body, wherein the applicator head comprises an applicator surface distally located in relation to the body and adapted for use in applying the substance to the site; and
a gas stream generator present near the applicator head for directing a stream of gas from the applicator head on to the site, wherein the site is dried during at least one of prior to, during and after an application of the substance,
wherein the stream of gas is directed via an outlet proximate to the applicator surface and the substance is applied via at least one of the application surface and a pad attached to the application surface.

2. The oral applicator of claim 1, wherein the body defines a housing for housing the gas stream generator.

3. The oral applicator of claim 2, wherein the applicator head is of elongate dimensions and is in a shape of an elbow.

4. The oral applicator of claim 3, wherein the gas stream generator comprises a fluid conduit from a source of gas under pressure to the outlet proximate to the applicator surface.

5. The oral applicator of claim 4, wherein the gas stream generator comprises a blower, wherein the blower comprises a fan and means for driving the fan.

6. The oral applicator of claim 5, wherein the fan driving means comprises a motor and a power source operatively connected to the motor, further wherein the blower comprises a power switch operable to switch power to the fan motor on and off.

7. The oral applicator of claim 6, wherein the applicator head is detachably connected to the body by a connector comprising a socket.

8. The oral applicator of claim 7, wherein the socket connects a fluid conduit from the fan of the gas stream generator to a fluid conduit in the applicator head leading to the outlet.

9. The oral applicator of claim 8, wherein the applicator surface comprises the pad comprising a resilient pad.

10. The oral applicator of claim 9, wherein the resilient pad is configured to absorb.

11. The oral applicator of claim 10, wherein the resilient pad comprises a contiguous surface.

12. The oral applicator of claim 11, wherein the contiguous surface is rounded.

13. The oral applicator of claim 12, wherein the resilient pad is configured to be detached from the applicator surface.

14. The oral applicator of claim 13, wherein the resilient pad is replaceable with another pad of a differing surface configuration.

15. The oral applicator of claim 14 further comprising an illuminator operable for illuminating the site to which a substance is to be applied using the applicator surface.

16. The oral applicator of claim 15, wherein the illuminator comprises a light pipe leading light from a light source to a light outlet on the applicator head.

17. The oral applicator of claim 16, wherein the gas stream generator comprises a reservoir of gas under pressure and means for allowing the gas to escape from the reservoir in a stream.

18. The oral applicator of claim 17, wherein the reservoir stores the gas in an aerosol form.

19. The oral applicator of claim 18, wherein the applicator head is detachably connected to the body.

20. A method of applying a substance to a site in a mouth cavity of a human or an animal, the method comprising:
providing an oral applicator comprising an applicator head extending from a body adapted for being held by a human hand, wherein the applicator head comprises an applicator surface distally located in relation to the body and adapted for use in applying the substance to the site, and a gas stream generator present near the applicator head for directing a stream of gas from the applicator head on to the site;
applying said substance to application surface of the applicator head;
directing a stream of the gas for drying the site, wherein the stream of gas is directed via an outlet proximate to the applicator surface; and
applying the substance from the applicator surface to the site for transferring the substance to the site.

21. The method of claim 20 further comprising directing the stream of the gas for drying the site during at least one of prior to application of the substance, during application of the substance to the site, and after application of the substance to the site.

22. The method of claim 21, wherein the body defines a housing for housing the gas stream generator, wherein the gas stream generator comprises a fluid conduit from a source of gas under pressure to the outlet proximate to the applicator surface.

23. The method of claim 22, wherein the gas stream generator comprises a blower housed within the body, wherein the blower is connected to be in fluid communication with the outlet, further wherein the blower comprises a fan and means for driving the fan.

24. The method of claim 23, wherein the substance is applied via at least one of the application surface and a pad attached to the application surface.

25. A kit for applying a substance to a site inside a mouth cavity of a human or an animal, the kit including an applicator apparatus comprising:
a body adapted for being held by a human hand;
an applicator head detachably connected to the body, wherein the applicator head comprises an applicator surface distally located in relation to the body and adapted for use in applying the substance to the site; and
a gas stream generator present near the applicator head for directing a stream of gas on to the site for drying the site, wherein the site may be dried before, during or after application of the substance, wherein the gas stream generator comprises a fluid conduit from a source of gas under pressure to an outlet proximate to the applicator surface.

26. The kit of claim 25 further comprising a plurality of applicator heads configured to interchangeably attach to the body.

27. The kit of claim 26 further comprising a plurality of applicator elements comprising interchangeable respective applicator surfaces for operatively fitting to the applicator head.

28. The kit of claim 27, wherein the applicator surfaces are of different respective configurations.

29. The kit of claim 28, wherein the gas stream generator comprises a blower, wherein the blower comprises a fan and means for driving the fan.

30. The kit of claim 29, wherein the applicator surface comprises a resilient pad configured to absorb, wherein the resilient pad comprises a contiguous surface.

31. The kit of claim 30, wherein the substance is applied via at least one of the application surface and the resilient pad attached to the application surface.

32. The kit of claim 31, wherein the resilient pad is configured to be detached from the applicator surface.

33. The kit of claim 32 further comprising an illuminator operable for illuminating the site to which a substance is to be applied using the applicator surface, wherein the illuminator comprises a light pipe leading light from a light source to a light outlet on the applicator head.

34. The kit of claim 33, wherein the stream of gas is directed via the outlet proximate to the applicator surface and the substance is applied via at least one of the application surface and resilient pad attached to the application surface.

35. The kit of claim 34 further comprising a rechargeable battery cell.
